(19) United States
(12) Patent Application Publication
(10) Pub. No.: US 2019/0104860 A1
(43) Pub. Date: Apr. 11, 2019

(54) COMPUTER-SHAPED MOTION BED SYSTEMS AND METHODS

(71) Applicant: J. Seth Blumberg, (US)

(72) Inventor: J. Seth Blumberg, Vail, CO (US)

(21) Appl. No.: 15/985,643

(22) Filed: May 21, 2018

Related U.S. Application Data
(60) Provisional application No. 62/508,418, filed on May 19, 2017.

(51) Int. Cl. A47C 31/12; A47C 23/34; A47C 27/16; A47C 31/00; G05B 19/042; A61F 5/56

(52) U.S. Cl. CPC A47C 31/123; A47C 23/34; A47C 27/16; G05B 2219/25252; G05B 19/042; A61F 5/56; G05B 2219/25257; A47C 31/003

(57) ABSTRACT

A computer-shaped motion bed system includes a mattress having embedded therein an array of disks, a sensor array for sensing body position on the mattress, an array of actuators, and an array of connectors for connecting each of a plurality of the disks embedded in the mattress to each of a plurality of the actuators. The system includes a controller having a processor coupled to a data store and in communication with the array of actuators, and programmed, upon receiving signals from the sensor array, to cause the a subset of the array of actuators to pull on a subset of the array of connectors, thereby pulling on a subset of the array of disks embedded in the mattress, so as to alter contours of a surface of the mattress, whereby the mattress is caused to increase comfort of a user, match contours of the user's body, or better support the user's body.

This application claims the benefit of U.S. Provisional Patent Application No. 62/508,418 filed May 21, 2017, the entire disclosure of which is incorporated herein by reference.

This application includes material which is subject to copyright protection. The copyright owner has no objection to the facsimile reproduction by anyone of the patent disclosure, as it appears in the Patent and Trademark Office files or records, but otherwise reserves all copyright rights whatsoever.

### FIELD

The present invention relates in general to the field of beds, and in particular to a novel mechanical and electrical systems for motion beds.

### BRIEF DESCRIPTION OF THE DRAWINGS

The foregoing and other objects, features, and advantages of the invention will be apparent from the following more particular description of preferred embodiments as illustrated in the accompanying drawings, in which reference characters refer to the same parts throughout the various views. The drawings are not necessarily to scale, emphasis instead being placed upon illustrating principles of the invention.

FIG. 1 shows a side view illustrating the invention according to a first embodiment.

FIG. 2 shows a side view illustrating the invention according to a second embodiment.

FIG. 3 shows a block diagram of a data processing system that can be used in various embodiments of the disclosed systems and methods.

FIG. 4 shows a block diagram of a user device.

### DETAILED DESCRIPTION

Reference will now be made in detail to the preferred embodiments of the present invention, examples of which are illustrated in the accompanying drawings. The following description and drawings are illustrative and are not to be construed as limiting. Numerous specific details are described to provide a thorough understanding. However, in certain instances, well-known or conventional details are not described in order to avoid obscuring the description. References to one or an embodiment in the present disclosure are not necessarily references to the same embodiment; and, such references mean at least one.

Reference in this specification to “an embodiment” or “the embodiment” means that a particular feature, structure, or characteristic described in connection with the embodiment is included in at least an embodiment of the disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The present invention is described below with reference to operational illustrations of methods and devices for providing motion beds. It is understood that each function of the operational illustrations, and combinations of functions, may be implemented by means of analog or digital hardware and computer program instructions. These computer program instructions may be stored on computer-readable media and provided to a processor of a general-purpose computer, special-purpose computer, ASIC, or other programmable data processing apparatus, such that the instructions, which execute via the processor of the computer or other programmable data processing apparatus, implements the functions/acts specified. In some alternate implementations, the functions noted may occur out of the order noted in the operational illustrations.

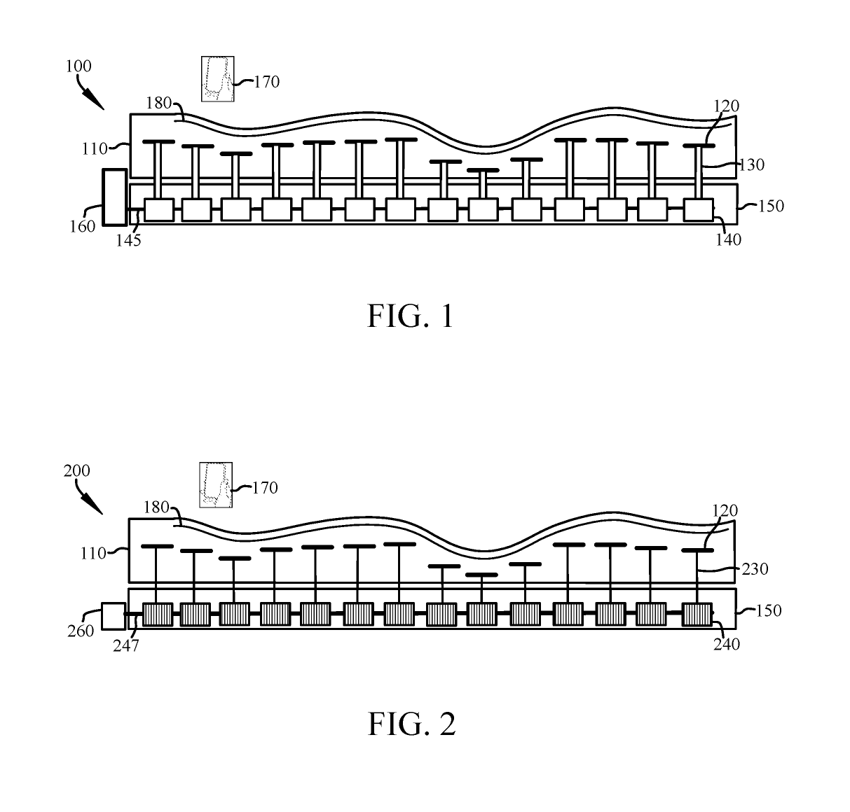

With reference to FIG. 1, in an embodiment, the present invention provides a computer-shaped motion bed system 100 that includes a mattress 110 having an array of rigid or semi rigid disks embedded therein. Each disk 120 is connected via a connecting means 130 to an actuator 140 in an array of actuators housed in an actuator housing 150. In embodiment, the actuator housing 150 is separate from the mattress 110. In another embodiment, the actuator housing is integrated into the mattress 110. The system 100 uses a sensor array 180, which may be in the form of a sheet of eTextile material, along with a controller 160 and a computer 170 (which may be a handheld device such as a smart phone or tablet, or a conventional laptop, desktop or server) to create a surface for supporting one or more persons' bodies in any position, including but not limited to laying down, sleeping, sitting, and/or reclining. The invention provides a system 100 that is able to change its shape in order to support the user's (or multiple users') body/bodies in a manner that is most preferred by the user(s) or that is shaped to the contours of the user's body when the user's body (or the bodies of multiple users at the same time) is in various positions.

One embodiment of the invention utilizes a mattress 110 comprising a memory foam (e.g., gel foam) material, water, hydraulic fluid, beads, crushed rubber or crushed foam, coil springs, air, gas, or any combination of these materials.

Via the use of the computer 170, the controller 160, and sensor array 180 for detecting the position of a body or part thereof, the contours of the mattress material (and/or box spring) can be altered to increase the comfort of the user and/or to match the contours of the user's body and/or to best support the body/bodies of the user.

For purposes of creating a custom-made pillow or configuring the bed, the contours of the user's body can be measured using, e.g., the user's impression into the memory foam mattress, a photograph or video of the user, or a wand or other scanner that is used to scan the user's body contours. In the case of a pregnant woman, this method can allow a pregnant woman to sleep on her stomach.

In an embodiment, the present invention provides a controller 160 and a series of actuators 140, 240, which may comprise more than twelve and preferably more than twenty four actuators, which together are capable of altering the shape or contours of various items including but not limited to various types of materials contained in a mattress, chair, seat, sofa, or recliner (or any combination of the above) including but not limited to material or supportive material of any type of seat, car seat, airplane seat, bus seat, other vehicular seat (all of which may herein sometimes be referred to as any of “chair” or “seat” or “sofa” or “recliner” or “Supportive Material”) which provides increased comfort level of the user or matching a prescription by a doctor or recommendation of a therapist.

In addition, in an embodiment, this invention includes systems and methods for altering or adjusting the shape of a supportive material using a novel design of a mattress material or box spring material.

The embedded disks 120 need not be disk-shaped, but can be any rigid or semi-rigid embedded material such as metal or plastic disks, or items of other shapes such as rectangles. The disks are embedded into a mattress 110 such as a memory foam mattress (or a mattress consisting of one or more other materials, or a chair or supportive material consisting of one or more other materials).

In an embodiment, a layer of discs 120 (which in this embodiment may or may not touch each other) are embedded inside a memory foam mattress, for example two inches below the top surface of the mattress (or seat, chair, recliner, sofa, car seat, airplane seat, vehicle seat, etc.). The disks 120 may comprise, e.g., metal, plastic, or other suitable material, preferably a rigid or semi-rigid material with enough strength and surface area to pull a section of the mattress downward or push it upward. If the discs are inserted into the mattress at a location that is not too close to the top surface of the mattress, the edges of the disks 120 are unlikely to cause discomfort to the user. In such implementation, the user's body (or users' bodies) can comfortably rest on memory foam without the user feeling the disks that are embedded below the surface of mattress. Each disk 120 is able to be pulled downwards by a connecting means. In FIG. 1, the connecting means 130 is a rigid member. In FIG. 2, an embodiment of the system 200 is shown wherein the connecting means 230 is a tether, for example a string, rope, or cable (any such tether herein sometimes referred to as the “tether” or the “cable”) that is tied to that particular disk. Each connecting means 230 connects at least one disc to a pulling device 240 that pulls the disk 120 downwards (for example, towards the ground) in order to create an indentation, void, or gap in the area of the top of the mattress 110, for example, above that particular disk. The pulling devices 240 may comprise a spindle around which the connecting means 230 winds. The pulling devices 240 may be individually driven under control of the controller, or multiple pulling devices may be driven by a single shaft transmission mechanism 240 as shown in FIG. 2.

When tension on the connecting means 230 is fully released, the disk 120 rises back upwards toward the position at which that part of the memory foam mattress naturally tends to return, e.g., a flat position at which it does not provide any positive or negative relief. The mattress 110 (which may be a memory foam mattress) has the natural tendency to return to it's normal (e.g., flat) shape when tension is released on the pulling device 230 that is attached to the disk 120. In an embodiment, only a flexible connecting means is provided and the disks 120 can only be pulled down or released upward. In other embodiments, such as that shown in FIG. 1, a rigid connecting means is provided so that the disk 120 can be pulled down and/or pushed up.

In the embodiment of FIG. 2, motor 260 and the driven transmission mechanism 247 comprising a rotating drive shaft parallel to the floor are provided. The transmission mechanism 247 may comprise a series of cams or other transmission means for providing upward and downward motion to the connecting means. A series of switches and/or clutches may be used to select which one(s) of the cams are engaged so as to selectively move one or more of the connecting means up or down.

As an alternative to memory foam, the mattress 110 may utilize coil springs that are pulled downward by a connecting means 230 such as a flexible cable, cord, wire, rod or bar. The connecting means 230 can be positioned to be located in the center of the spring and attached to a disc that's located at the top of the spring. When the connecting means 230 is pulled downwards, in turn the connecting means pulls the disk downwards and the disk pulls the spring downwards (effectively compressing the spring), thus creating a negative relief or depression in the surface of the mattress at that particular location.

When tension on the connecting means 130 or 230 is applied downwards in various increments/amounts, then the disk is pulled downwards and that creates a depression or negative relief at the surface of the mattress above that particular disk and that part of the surface of the mattress 110 will have a negative relief with a depth that is proportional to the amount of tension pulling downwards on the connecting means 130 or 230. In this case, the mattress 110 is at less than full thickness at that location because that part of the mattress is being pulled downwards by the connecting means and disk.

The pulling device 240 may comprise, for example, a motorized spindle that winds a cable or other flexible connecting means in order to effectively pull at least one disc down toward the floor.

The motor 260 (or plurality of motors) and transmission mechanism 247 attached to each pulling device are controlled by the controller 160. The controller is electrically connected to the actuators by wired or wireless connection 145. The computer 170 may be used to provide an interface to the controller.

As a result, the contours of a mattress, chair, bed or supportive material can be controlled and shaped to match the contours of the user's body.

As an alternative to using a flexible connecting means (e.g., a cable), a rigid connecting means such as that shown at 130 in FIG. 1 can be used. The connecting means 130 may be any type that is useful for connecting the disk 120 with the actuator 140, such as a rod or bar. The rigid connecting means allows the disc not only to be pulled downwards but also to be pushed upwards in order to create a positive relief at the surface of the mattress above that particular disc. In this embodiment, a wall could be created by pulling one such connecting means 130 downward and pushing an adjacent connecting means upward. For example, a six-inch wall could be created by pulling one connecting means 130 (or 230) downward by three inches and pushing an adjacent connecting means upward by three inches. Both a flexible connecting means and a rigid connecting means may be attached to each disk 120. Also, the function of either the flexible and/or the rigid connecting means may be provided by magnets or a system for magnetic levitation to attract, repel, or otherwise move the disc to achieve a differing height or elevation of the surface of the bed at various locations. An array of actuators may be provided as the rigid connecting means, with each actuator connected to one of the disks or each actuator selectively connected to one or more of the disks. A ratchet mechanism or other locking or retaining mechanism may be provided in association with the actuator 140 and/or connecting means 130. After a movement of a disc is made, the ratchet mechanism or other locking or retaining mechanism may be used to hold the disc at the new height until it is moved again.

The system 100 can be configured such that the user can communicate with the controller 160 in a myriad of ways, including the following. The user can use a code communicated by tapping the user's fingers or hand or foot or toes on the mattress in a manner that communicates that the mattress should change shape. For example, tapping at a particular location could be sensed by the sensor array 180 and this can be read by the controller 160 which causes the bed to be either be lowered or raised at the particular location that was tapped. The sensor array 180 may be a sheet of eTextile fabric, pressure sensors, or acoustic sensors in the mattress that are used to detect such tapping. The sheet of eTextile fabric may be of the type that uses yarn coated with Piezo-electric polymer that changes resistance when compressed. Wires traversing the fabric, typically at right angles, sense this resistance when applying voltage across the two wires. The sensor array 180 may be capable of sensing acceleration, position, or curvature. The actuators 140 may comprise electro-magnetic devices such as solenoids, screw-type actuators, or scissor-type actuators. The actuators 140 or the motor 260 may comprise an electric motor such as a stepper motor, which may also be the screw type or scissor type. The actuator throw is preferably greater than two inches and may be as long as twelve inches in some embodiments or twenty-four inches in other embodiments. In an embodiment, the actuator throw is greater than one and a half inches. The processor of the controller 160 may comprise a microcontroller, DSP processor, a computer, or other processing device. Algorithms can be provided in the controller for sensing sleeping posture and pressure points, and for monitoring and mitigating sleep apnea by observing the initial signs of sleep apnea and moving the sleeper to their side or applying another mitigating strategy.

In an embodiment, the user can use voice commands to cause the bed to change shapes to match the contours of a particular sleeping position to which the user would like the bed to conform. For example, the user can speak the word “side” to cause the bed to change to a shape that supports the user in a side sleeping position. As another example, the user can speak the term “roll over” to cause the bed to change shape in a manner which urges the speaker to roll over. This voice control can be made via an intelligent automated personal assistant or smart personal agent. Such automated personal assistants include, e.g., Apple's Siri, Google Assistant, Amazon Echo, Microsoft Cortana, or the like. The bed described herein may be controlled by a smart watch or other wearable device capable of receiving commands from a user and wirelessly communicated them to the controller or to an automated personal assistant. The bed disclosed herein may be configured to be a computer appliance or smart connected product on the Internet of Things.

In an embodiment, mattresses described herein may include a channel or tube as a way of transporting a cooling liquid. The liquid may be pumped through the tube or channel, and may be pumped via a compressor. The sensors may include temperature sensors, which may be monitored by the controller and used to determine which part of the sleeper's body requires cooling. The cooling liquid may then be directed to a corresponding area of the mattress. Alternatively, a layer of cooling material, such as a liquid gel or pebbles of heat-absorbing material, may be provided below the foam layer in the mattress.

The inventions of this disclosure can be combined with functions and hardware disclosed in the present inventor's U.S. patent application Ser. No. 11/851,204, the entire disclosure of which is incorporated herein by reference. For example, a doctor's prescription can be used to indicate the shape of the bed. In one such embodiment, the bed can automatically contort itself to assume the shape that would cause the user's vertebrae to be placed in an ergodynamic position. For example, the bed can automatically contort itself to assume the shape that allows for a pregnant woman to sleep on her stomach by creating a bulge in the bed for the pregnant woman's abdomen. As another example, the bed could create negative relief to allow for a user's feet to stick down into the bed to simulate the experience of the user sleeping on his/her stomach with his/her feet hanging off of the bed.

As a further example, the sensors and/or the controller can be configured to locate the user's body position/location on the bed.

In an embodiment, the bed can create or include a negative relieve that receives the user's face. For example, the bed could contort itself to create a negative relief around the user's face to simulate the user sleeping face down on a massage table with the user's head facing straight down toward the ground and the bed supporting the user's forehead and sides of the head so that there is a hole below the user's nose, mouth, eyes, etc. and the bed would create a hole below the user's face that allows for replenishing of with fresh air into the hole (below the face). The fresh air could flow through a channel that's created by the contours of the bed. Optionally there could be a fan to encourage air flow through the channel to the hole that's located below the user's face. Optionally, there could be a television monitor located in the hole so that the user could optionally watch some videos.

Another invention disclosed herein relates to a custom-made pillow that can be used to increase comfort and/or support a person when sleeping or sitting in any type of chair. In one embodiment, the pillow is custom-made to match the contours of the user's body from the user's head (or beyond the user's head) to the user's toes (or beyond the user's toes) when the user is laying in any one (or more) particular position. For example, the pillow can be created to match the contours of the user's body when the user is laying flat on the user's back, when the user is laying flat on the user's stomach, when the user is laying flat on the user's right side, when the user is laying flat on the user's left side, when the user is laying in any particular position in between being flat on the user's back and flat on the user's stomach.

The custom-made pillow may be designed to support the user in a position that is in between being flat on the user's back and flat on the user's stomach. There are a myriad of examples of positions whereby the user is laying in a particular position in between being flat on the user's back and flat on the user's stomach, including:
The user lays halfway (or any percentage) between laying on the user's right side and laying on the user's back; or
The user lays halfway (or any percentage) between laying on the user's right side and laying on the user's stomach; or
The user lays halfway (or any percentage) between laying on the user's left side and laying on the user's back; or
The user lays halfway (or any percentage) between laying on the user's left side and laying on the user's stomach; or
The user lays in any of myriad partial fetal positions to full fetal position on the user's left side or any of myriad positions between the left side and laying flat on the user's back.
The user lays in any of myriad partial fetal positions to full fetal position on the user's right side or any of myriad positions between the right side and laying flat on the user's back.

The custom-made pillow may be contoured so as to match the shape of a pregnant woman.

The custom-made pillow may be created in a myriad of ways/methods and from a myriad of one or more materials. The custom pillow may be printed by a three-dimensional printer. The user might lay down in a mold creating device.

Motion Bed Design for Alleviating Pressure from Bed Sores and Other Skin Issues

The cells can be lowered in the area where one or more skin issue is located. The term skin issue as used herein includes a bed sore, decubitus, pressure sore, skin contusion, cut on the skin, skin abrasion, or other problem with the skin. In an embodiment, the bed can determine the location of the skin issue.

Further, in order to decrease the chances of a new skin issue due to long periods of immobility on a bed, chair, wheel chair recliner, sofa, car seat, or vehicle seat, etc., the cells of the bed (or the various parts of the surface of, for example, a memory foam bed that are controlled by a connecting means pulling down on, for example, a cable) can be alternately raised and lowered in a manner that avoids long periods of time with pressure on any one particular area of skin.

Further, the surface of the bed can be positioned to allow air flow for any area where there is an existing skin issue. The air flow can increase the chance of healing of the skin issue. The surface of the bed can be positioned to allow for a channel in which air can flow to the skin issue, and optionally a fan can increase the air flow through the channel to the area where the skin issue is located.

In an embodiment, the controller can be configured such that the position of the bed can be controlled by an app running on a smart phone or other computing device such as a laptop or desktop computer.

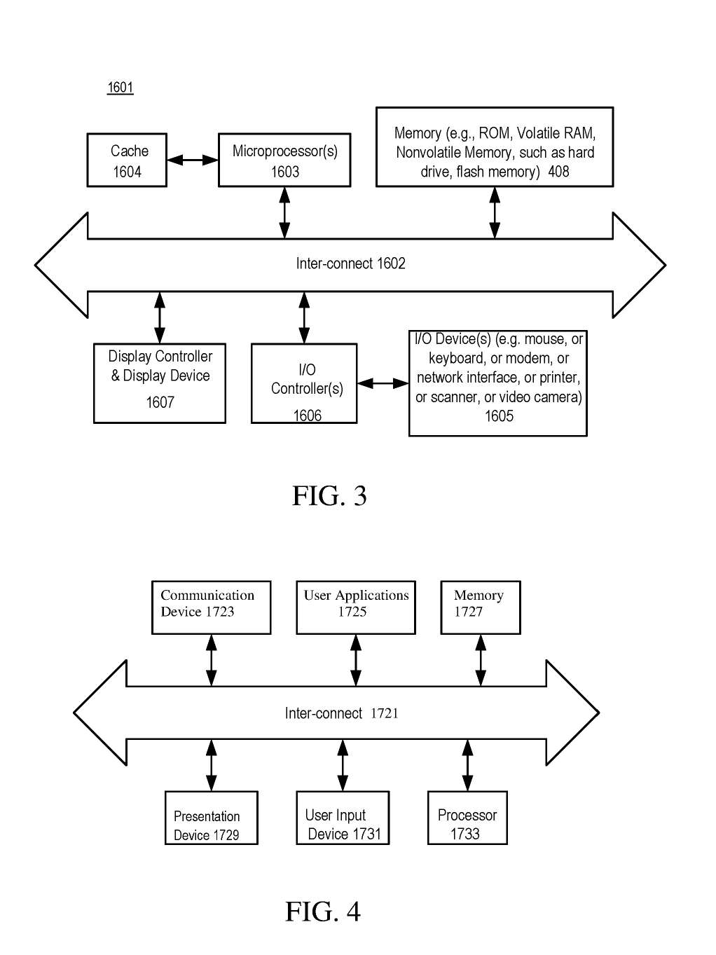

FIG. 3 shows a block diagram of a data processing system that can be used as the controller 160 (FIG. 1) in various embodiments of the disclosed systems and methods. While FIG. 3 illustrates various components of a computer system, it is not intended to represent any particular architecture or manner of interconnecting the components. Other systems that have fewer or more components may also be used.

In FIG. 3, the system 1601 includes an inter-connect 1602 (e.g., bus and system core logic), which interconnects a microprocessor(s) 1603 and memory 1608. The microprocessor 1603 is coupled to cache memory 1604 in the example of FIG. 3.

The inter-connect 1602 interconnects the microprocessor(s) 1603 and the memory 1608 together and also interconnects them to a display controller and display device 1607 and to peripheral devices such as input/output (I/O) devices 1605 through an input/output controller(s) 1606. Typical I/O devices include mice, keyboards, modems, network interfaces, printers, scanners, video cameras and other devices that are well known in the art.

The inter-connect 1602 may include one or more buses connected to one another through various bridges, controllers and/or adapters. In one embodiment the I/O controller 1606 includes a USB (Universal Serial Bus) adapter for controlling USB peripherals, and/or an IEEE-1394 bus adapter for controlling IEEE-1394 peripherals.

The memory 1608 may include ROM (Read-Only Memory) and volatile RAM (Random Access Memory), and non-volatile memory, such as hard drive, flash memory, etc. Volatile RAM is typically implemented as dynamic RAM (DRAM) that requires power continually in order to refresh or maintain the data in the memory. Non-volatile memory is typically a magnetic hard drive, a magnetic optical drive, or an optical drive (e.g., a DVD RAM), or other type of memory system which maintains data even after power is removed from the system. The non-volatile memory may also be a random access memory. The non-volatile memory can be a local device coupled directly to the rest of the components in the data processing system. A non-volatile memory that is remote from the system, such as a network storage device coupled to the data processing system through a network interface such as a modem or Ethernet interface, can also be used.

In an embodiment, one or more servers supporting the platform are implemented using one or more data processing systems as illustrated in FIG. 3. In an embodiment, user devices such as those used to access the user interfaces described above are implemented using one or more data processing system as illustrated in FIG. 3.

In some embodiments, one or more servers of the system illustrated in FIG. 3 are replaced with the service of a peer-to-peer network or a cloud configuration of a plurality of data processing systems, or a network of distributed computing systems. The peer-to-peer network, or cloud-based server system, can be collectively viewed as a server data processing system.

Embodiments of the system disclosed above can be implemented via the microprocessor(s) 1603 and/or the memory 1608. For example, the functionalities described above can be partially implemented via hardware logic in the microprocessor(s) 1603 and partially using the instructions stored in the memory 1608. Some embodiments are implemented using the microprocessor(s) 1603 without additional instructions stored in the memory 1608. Some embodiments are implemented using the instructions stored in the memory 1608 for execution by one or more general-purpose microprocessor(s) 1603. Thus, the disclosure is not limited to a specific configuration of hardware and/or software.

FIG. 4 shows a block diagram of a user device. In FIG. 4, the user device includes an inter-connect 1721 connecting a communication device 1723, such as a network interface device, a presentation device 1729, such as a display screen, a user input device 1731, such as a keyboard or touch screen, user applications 1725 implemented as hardware, software, firmware or a combination of any of such media, such various user applications (e.g. apps), a memory 1727, such as RAM or magnetic storage, and a processor 1733 that, inter alia, executes the user applications 1725.

In one embodiment, the user applications implement one or more user interfaces displayed on the presentation device 1729 that provides users and the system the capabilities to, for example, access a Wide Area Network (WAN) such as the Internet, and display and interact with user interfaces provided by the platform, such as, for example the user interfaces described above in this disclosure. In an embodiment, users use the user input device 1731 to interact with the device via the user applications 1725 supported by the device.

While some embodiments can be implemented in fully functioning computers and computer systems, various embodiments are capable of being distributed as a computing product in a variety of forms and are capable of being applied regardless of the particular type of machine or computer-readable media used to actually effect the distribution.

At least some aspects disclosed can be embodied, at least in part, in software. That is, the techniques may be carried out in a special purpose or general purpose computer system or other data processing system in response to its processor, such as a microprocessor, executing sequences of instructions contained in a memory, such as ROM, volatile RAM, non-volatile memory, cache or a remote storage device. Functions expressed in the claims may be performed by a processor in combination with memory storing code and should not be interpreted as means-plus-function limitations.

Routines executed to implement the embodiments may be implemented as part of an operating system, firmware, ROM, middleware, service delivery platform, SDK (Software Development Kit) component, web services, or other specific application, component, program, object, module or sequence of instructions referred to as “computer programs.” Invocation interfaces to these routines can be exposed to a software development community as an API (Application Programming Interface). The computer programs typically comprise one or more instructions set at various times in various memory and storage devices in a computer, and that, when read and executed by one or more processors in a computer, cause the computer to perform operations necessary to execute elements involving the various aspects.

A machine-readable medium can be used to store software and data which when executed by a data processing system causes the system to perform various methods. The executable software and data may be stored in various places including for example ROM, volatile RAM, non-volatile memory and/or cache. Portions of this software and/or data may be stored in any one of these storage devices. Further, the data and instructions can be obtained from centralized servers or peer-to-peer networks. Different portions of the data and instructions can be obtained from different centralized servers and/or peer-to-peer networks at different times and in different communication sessions or in a same communication session. The data and instructions can be obtained in entirety prior to the execution of the applications. Alternatively, portions of the data and instructions can be obtained dynamically, just in time, when needed for execution. Thus, it is not required that the data and instructions be on a machine-readable medium in entirety at a particular instance of time.

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., Compact Disk Read-Only Memory (CD ROMS), Digital Versatile Disks (DVDs), etc.), among others.

In general, a machine-readable medium includes any mechanism that provides (e.g., stores) information in a form accessible by a machine (e.g., a computer, network device, personal digital assistant, manufacturing tool, any device with a set of one or more processors, etc.).

In various embodiments, hardwired circuitry may be used in combination with software instructions to implement the techniques. Thus, the techniques are neither limited to any specific combination of hardware circuitry and software nor to any particular source for the instructions executed by the data processing system.

As used herein, ordinal terms such as first and second are not intended, in and of themselves, to imply sequence, time or uniqueness, but rather are used to distinguish one claimed construct from another. In some uses where the context dictates, these terms may imply that the first and second are unique. For example, where an event occurs at a first time, and another event occurs at a second time, there is no intended implication that the first time occurs before the second time. However, where the further limitation that the second time is after the first time is presented in the claim, the context would require reading the first time and the second time to be unique times. Similarly, where the context so dictates or permits, ordinal terms are intended to be broadly construed so that the two identified claim constructs can be of the same characteristic or of different characteristic.

While some embodiments can be implemented in fully functioning computers and computer systems, various embodiments are capable of being distributed as a computing product in a variety of forms and are capable of being applied regardless of the particular type of machine or computer-readable media used to actually effect the distribution.

The above embodiments and preferences are illustrative of the present invention. It is neither necessary, nor intended for this patent to outline or define every possible combination or embodiment. The inventor has disclosed sufficient information to permit one skilled in the art to practice at least one embodiment of the invention. The above description and drawings are merely illustrative of the present invention and that changes in components, structure and procedure are possible without departing from the scope of the present invention as defined in the following claims. For example, elements and/or steps described above and/or in the following claims in a particular order may be practiced in a different order without departing from the invention. Thus, while the invention has been particularly shown and described with reference to embodiments thereof, it will be understood by those skilled in the art that various changes in form and details may be made therein without departing from the spirit and scope of the invention.

What is claimed is:

1. A computer-shaped motion bed system, comprising:
a mattress having embedded therein an array of disks;
a sensor array for sensing body position on the mattress;
an array of actuators;
an array of connectors for connecting each of a plurality of the disks embedded in the mattress to each of a plurality of the actuators;
controller comprising a processor coupled to a data store and in communication with the array of actuators, and programmed, upon receiving signals from the sensor array, to cause a plurality of actuators in the array of actuators to pull on the a plurality of connectors in the array of connectors, thereby pulling on the array of a plurality of disks in the array of disks embedded in the mattress, so as to alter contours of a surface of the mattress, whereby the mattress is caused to increase comfort of a user, match contours of the user's body, or better support the user's body.

2. The computer-shaped motion bed system of claim 1, wherein the mattress having the disks embedded therein comprises a memory foam mattress.

3. The computer-shaped motion bed system of claim 2, wherein the memory foam mattress, array of disks, array of connectors, and array of actuators are configured such that when tension on a particular connector in the array is released, a disk connected to the particular connector rises back upward toward a position at which the memory foam mattress tends to return.

4. The computer-shaped motion bed system of claim 1, wherein the array of actuators comprises an array of at least twenty-four actuators.

5. The computer-shaped motion bed system of claim 1, wherein the array of disks comprises disks that are circular.

6. The computer-shaped motion bed system of claim 1, wherein the array of disks comprises disks that are non-circular.

7. The computer-shaped motion bed system of claim 1, wherein a plurality of connectors in the array of connectors are rigid and are configured to both pull and push a corresponding plurality of disks in the array of disks embedded in the mattress.

8. The computer-shaped motion bed system of claim 1, wherein a plurality of connectors in the array of connectors comprises tethers and the array of actuators comprises an array of pulling devices configured to each pull an associated tether, thereby pulling downward a disk in the array of disks embedded in the mattress.

9. The computer-shaped motion bed system of claim 8, wherein the tethers each comprise a cable and the actuators each comprise a motorized spool onto which one of the cables is wound.

10. The computer-shaped motion bed system of claim 1, wherein the array of connectors, the array of disks, and the array of actuators are configured to raise and lower the disks through magnetic levitation.

11. The computer-shaped motion bed system of claim 1, further comprising a locking or retaining mechanism operatively connected to the connector and configured to hold the disk at a raised or lowered height.

12. The computer-shaped motion bed system of claim 1, wherein the sensor array comprises a sheet of e-textile fabric.

13. The computer-shaped motion bed system of claim 1, wherein the sheet of e-textile fabric comprises yarn coated with Piezo-electric polymer that changes resistance when compressed.

14. The computer-shaped motion bed system of claim 1, wherein the sensor array comprises an acoustic sensor.

15. The computer-shaped motion bed system of claim 1, wherein the sensor array and controller are configured to sense tapping at a particular location on the mattress and raise or lower a portion of the surface of the mattress at the particular location in response to said tapping.

16. The computer-shaped motion bed system of claim 1, wherein the sensor array and controller are configured to sense tapping and change a shape of the contours of the surface of the mattress in response to the tapping.

17. The computer-shaped motion bed system of claim 1, wherein a throw of each actuator in the array of actuators is greater than one and a half inches.

18. The computer-shaped motion bed system of claim 1, wherein a throw of each actuator in the array of actuators is greater than two inches.

19. The computer-shaped motion bed system of claim 1, wherein the processor is further programmed to run an algorithm that senses sleeping posture and pressure points.

20. The computer-shaped motion bed system of claim 1, wherein the processor is further programmed to run an algorithm that monitors and mitigates sleep apnea by observing the initial signs of sleep apnea and applying a mitigating strategy.

21. The computer-shaped motion bed system of claim 20, wherein the mitigating strategy is urging the user onto his or her side.

22. The computer-shaped motion bed system of claim 20, wherein the array of actuators comprises an array of more than twenty-four actuators.
